(19) United States
(12) Patent Application Publication
(10) Pub. No.: US 2015/0297504 A1
(43) Pub. Date: Oct. 22, 2015

(54) COSMETIC USE OF A CAROB SEED EXTRACT AS A SLIMMING ACTIVE AGENT

(71) Applicants: Jean-Marie BOTTO, (US); ISP INVESTMENTS INC., Wilmington, DE (US)

(72) Inventors: Jean-Marie BOTTO, Garbejaire (FR); Nouha DOMLOGE, Valbonne (FR); Frederique PORTOLAN, Valbonne (FR)

(21) Appl. No.: 14/648,940

(22) PCT Filed: Dec. 10, 2013
(86) PCT No.: PCT/FR2013/053021

§ 371 Date: Jun. 2, 2015

(30) Foreign Application Priority Data
Dec. 11, 2012 (FR) 1203363

(51) Int. Cl. A61K 8/97; A61K 8/64; A61Q 19/06

(52) U.S. Cl. CPC A61K 8/97; A61Q 19/06; A61K 8/645

(57) ABSTRACT

Cosmetic methods for obtaining a slimming effect are disclosed. The methods include providing a composition containing a carob (Ceratonia siliqua L.) seed extract as a slimming agent and a physiologically acceptable medium, and topically applying the composition on at least a portion of the body or face. The topical application of the composition includes the application of an effective amount of the composition that increases the expression of aquaglyceroporins, promotes lipolysis, and/or reduces localized excess body fat.

### FIELD OF THE INVENTION

This invention concerns the field of cosmetics and more specifically the field of cosmetic weight-loss methods. It relates to the cosmetic use of a carob germ extract (Ceratonia siliqua L.) as a slimming agent. The invention also relates to the cosmetic use of the combination of a carob germ extract for increasing the expression of aquaglyceroporins and promoting the elimination of triglycerides contained in adipocytes.

The invention also relates to a method of cosmetic care including the topical application, on at least a portion of the skin of the body or the face, of a carob germ (Ceratonia siliqua L.) in a composition containing a physiologically acceptable medium, in order to obtain a slimming effect, and more specifically to reduce localized excess body fat.

### BACKGROUND OF THE INVENTION

The subcutaneous adipose tissue is located at the hypodermis. It is a type of connective tissue where adipocytes are predominant, organized in lobes around 5 mm in diameter, separated by fine connective bridges. Each adipocyte contains a voluminous lipid vacuole containing essentially triglycerides and having a diameter that may range from 40 to 120 μm.

Adipose tissue may be considered to be a dynamic reservoir, constantly being renewed, balancing the dietary intake with the energy requirements of the body. Thus, adipocytes ensure the synthesis, accumulation and release of lipids.

Lipid synthesis, or lipogenesis, originates with triglycerides of dietary origin and glucose. Conversely, the triglycerides stored in the adipocytes may be hydrolyzed, during lipolysis, to release fatty acids, glycerol and glycerol mono- and diesters.

The non-esterified fatty acids thus released may circulate in the blood and then be available for the energy requirements of other cells of the body, or be quickly reused by the adipocyte so as to generate, again, triglycerides by lipogenesis.

If a sustained imbalance occurs in the body promoting lipogenesis, the quantity of lipids stored in the adipocytes increases, leading to hyperplasia of the mass of body fat and more specifically to the appearance of localized excess body fat. In fact, in human adults, under the effect of sex hormones, the adipose tissue is distributed differently according to sex and forms the silhouette. Adipose tissue accumulates in the chest, on the hips, the buttocks and the thighs in women, and on the nape and shoulders in men. In addition, localized excess body fat is often associated with modifications in the skin, which develops a dimpled or “orange-peel” appearance. This localized excess body fat is currently considered to be unattractive, and people affected may want to improve the appearance of their skin and silhouette using cosmetic methods.

Numerous active agents having an action on lipolysis or lipogenesis, intended for slimming effect, have thus been identified. Among them, the following may be cited:
Xanthine bases (xanthine derivatives), such as theophylline, caffeine, theobromine (described in patents FR 2 609 395, FR 2 674 01), used for their action promoting the lipolytic activity of fat cells.
Synthetic peptides, such as the peptide of sequence Arg-Gly-Ser-NH2 (described in patent FR 2 858 769), or the peptide of sequence Pro-Leu-Asp-Thr-Ala-Lys-Val-Arg-Leu-Gln (described in patent FR 2 879 924) used for their action in the decoupling between coenzyme reoxidation and phosphorylation of ADP into ATP in the mitochondria.
Plant extracts, such as marine algae extract of the Palmaria or Rhodymenia genus (described in patent FR 2 887 447), gingko biloba extracts (see patent FR 2 669 537), or soy flavones or isoflavones (described in patent WO 01/64177).

However, these products generally have moderate or limited efficacy over time. It is therefore important to provide new active cosmetic agents having remarkable efficacy as slimming active agents.

The solution to the technical problem addressed lies in the cosmetic use of the association of a carob germ extract. The inventors have indeed demonstrated that a carob germ extract acts on aquaglyceroporins, thereby promoting the transport of glycerol released during lipolysis, from the adipocyte.

Aquaporins are a class of transmembrane proteins carrying water and small molecules in solution, between the cells and the internal medium. Aquaporins may be classified into two distinct sub-groups: aquaporins enabling only the transport of water, and aquaglyceroporins which enable, in addition to the transport of water, the transport of glycerol.

In this second sub-group, aquaglyceroporin 7 was identified in the membrane of human adipocytes and plays an important role in the metabolism of reserve fats (Mariko Hara-Chikuma et al. Progressive adipocyte hypertrophicity in aquaporin-7 deficient mice, J. Biol. Chem., vol. 280, no. 16, Apr. 22, 2005).

The particular properties of aquaglyceroporins therefore makes them beneficial biological targets for promoting the elimination of lipids contained in the adipocytes.

“Elimination of lipids” refers to the phenomenon of lipolysis leading to the export of glycerol from the adipocyte cell.

The invention and the resulting advantages will be better understood upon reading the description.

### DESCRIPTION OF THE INVENTION

This invention first relates to the cosmetic use of a carob germ extract (Ceratonia siliqua L.) as a slimming active agent.

The association of the carob germ extract and caffeine or a derivative thereof, used according to the invention, provides in particular the following advantages:
it increases the expression of aquaglyceroporins,
it promotes lipolysis, the elimination of lipids and the export of glycerol from the adipocytes,
it reduces localized excess body fat.

Thus, “slimming active agent” in the sense of this invention refers to a carob germ extract used to reduce excess localized body fat, considered to be unattractive and often associated with a dimpled appearance of the skin.

Carob seed (plant of the Ceratonia genus), and more specifically the endosperm fraction of said seed, is widely used for its rich galactomannan content in the food industry under the name “carob gum”. The germ is the portion of the seed highest in protein and can easily be isolated.

A carob germ protein extract, obtained by enzymatic hydrolysis, has already been described (J. Parrado et al., Bioresource Technology 99, 2008). Nevertheless, this extract has too high a concentration of phytohormones, an endocrine disrupter, to be used in cosmetics.

To carry out the invention, any extraction or purification method known to a person skilled in the art may be used, such as, for example, the method described in the European patent application EP 0 689 771.

“Peptide extract” refers to a mixture of compounds predominantly represented by peptide compounds, in solution in a large volume of water or other solvents, polar or a mixture of these solvents.

“Peptide compounds” refers to protein fragments and peptides present in the peptide extract according to the invention.

“Topical application” refers to the application or spreading of the active agent according to the invention, or a composition containing it, on the surface of the skin or a mucous membrane.

“Physiologically acceptable” means that the active agent according to the invention, or a composition containing it, is suitable for coming into contact with the skin or a mucous membrane without causing reactions of toxicity or intolerance.

“Physiologically acceptable” refers to any compound adapted to come into contact with the skin or a mucous membrane without causing reactions of toxicity or intolerance.

“Excess localized body fat” refers to a hypertrophied area of the subcutaneous adipose tissue, which may have an “orange peel” appearance.

In the description below, the terms “slimming active agent” and “carob germ extract” will be used interchangeably.

The active agent according to the invention may be obtained by extracting proteins of plant origin, followed by a controlled hydrolysis that releases the biologically active peptide compounds.

The use of peptide extracts, and in particular peptide extracts of low molecular weight, has numerous advantages in cosmetics. Aside from the fact of generating peptide compounds that did not previously exist in the starting protein mixture, hydrolysis and purification make it possible to obtain mixtures with greater stability, compositions that are easier to reproduce and not causing allergic reactions in cosmetics.

To carry out the extraction, carob seed germ (plant of the Ceratonia genus) is used. Any extraction or purification method known to a person skilled in the art may be used to prepare the extract according to the invention. For example, controlled hydrolysis makes it possible to release peptide compounds. It is possible, but unnecessary in order to carry out the invention, either to extract the proteins concerned and then hydrolyze them, or to perform the hydrolysis on a raw extract and then to purify the peptide compounds.

In a first step, the germ contained in the seeds is ground so as to obtain a powder or flour. The powder thus obtained may first be treated by a cellulase in order to promote the elimination of sugars, and in particular insoluble polysaccharides.

Then, the extraction of the proteins of the germ is performed according to the modified conventional process (Osborne, T. B., The Vegetable Proteins, 2nd Edition. Longmans, Green and Co., London, 1924); the ground carob germ is suspended in an alkaline solution containing an insoluble adsorbent polyvinylpolypyrrolidone (PVPP) product (0.01-20%); in fact, it is known that subsequent hydrolyses and purifications are facilitated by this means. In particular, the concentration of phenol substances interacting with the proteins is clearly reduced. The proteins can then be precipitated by varying the ionic strength or by acidifying the medium, thereby making it possible to eliminate the soluble components and the nucleic acids. The precipitate is then washed with an organic solvent such as, for example, ethanol or methanol, then the solvent is evaporated by vacuum drying. The protein-rich precipitate is placed in solution in water or another solvent and thus constitutes a more purified form of the extract.

The extraction may also be performed in a neutral or acid medium, still in the presence of polyvinylpolypyrrolidone. After a filtration step, the precipitation step is then carried out with a conventional precipitation agent such as salts (sodium chloride, ammonium sulfate) or an organic solvent (alcohol, acetone). The precipitate obtained may be separated from the precipitation agents by dialysis after being placed in solution in water or another solvent.

The soluble fraction, containing proteins, carbohydrates and possibly fats, is collected after the centrifugation and filtration step. This raw solution is then hydrolyzed under controlled conditions in order to generate soluble peptides. Hydrolysis is defined as being a chemical reaction involving the cleaving of a molecule by water, said reaction being capable of being produced in a neutral, acid or basic medium. According to the invention, hydrolysis is performed chemically and/or advantageously by proteolytic enzymes among which plant-based endoproteases may be cited (papain, bromelin, ficain).

According to a first embodiment of the invention, the slimming active agent includes the hydrolyzed carob germ extract obtained in this step.

For the same reasons as above, i.e. the elimination of polyphenol substances, a quantity of polyvinylpolypyrrolidone may be added to the reaction medium during this step of controlled hydrolysis. The extract obtained may again be purified in order to select the low-molecular-weight peptide compounds. Fractionation may advantageously be performed by ultra-filtration and/or by a chromatographic method.

Then a phase of dilution in water or in any mixture of solvents containing water is performed. Thus, according to an advantageous embodiment of the invention, the carob germ extract according to the invention is advantageously diluted in one or more physiologically acceptable solvents, such as water, glycerol, ethanol, propanediol, butylene glycol, dipropylene glycol, ethoxylated or propoxylated diglycols, cyclic polyols or any mixture of these solvents. The diluted carob germ extract is then sterilized by ultra-filtration.

Thus, according to an advantageous embodiment of the invention, the carob germ extract is diluted in one or more physiologically acceptable solvents, such as water, glycerol, ethanol, propanediol, butylene glycol, dipropylene glycol, ethoxylated or propoxylated diglycols, cyclic polyols or any mixture of said solvents.

After this dilution, a peptide extract is obtained, which is characterized by a dry weight of 2 to 5 g/kg, a peptide compound concentration of 1 to 10 g/l, preferably 1.5 to 3.5 g/l, a sugar concentration of 0.05 to 1 g/l, preferably 0.1 to 0.3 g/l and a polyphenol concentration of less than 1% with respect to the dry weight.

Thus, according to an advantageous embodiment of the invention, the carob germ extract has a dry weight of 2.5 g/kg and contains between 1.5 and 3.5 g/l of peptide compounds.

The physicochemical characteristics and the protein and peptide compound content of the extract obtained according to the invention are qualitatively and quantitatively analyzed, according to conventional techniques well known to a person skilled in the art.

The extract obtained is comprised of peptides having a molecular weight below 5 kDa and is characterized by a sugar concentration below 15% and a polyphenol concentration below 1% with respect to the dry weight.

Thus, according to an advantageous embodiment of the invention, the carob germ extract is a peptide extract in which the peptide compounds have a molecular weight below 5 kDa.

This invention also relates to the use of a carob germ extract in order to increase the expression of aquaglyceroporins and more specifically aquaglyceroporin 7.

The characteristic molecular activity of the invention is defined in vitro by the capacity of the slimming active agent to increase the expression of aquaglyceroporins, either by increasing the protein synthesis of aquaglyceroporins (by direct or indirect modulation of the gene expression of aquaglyceroporins), or by other biological processes such as the stabilization of the aquaglyceroporin protein or the stabilization of messenger RNA transcripts.

Preferably, according to this invention, aquaglyceroporin is aquaglyceroporin 7.

This invention also relates to the use of a carob germ extract in order to promote lipolysis, the elimination of lipids and the export of glycerol from the adipocytes.

The characteristic biological activity of the invention is defined in vitro by the capacity of the slimming active agent to reduce the size and number of lipid droplets in the adipocytes.

This invention also relates to a cosmetic care method including the topical application, on at least a portion of the skin of the body or face, of a carob germ extract (Ceratonia siliqua L.) in a composition containing a physiologically acceptable medium, in order to obtain a slimming effect, and more specifically in order to reduce excess localized body fat.

Advantageously, the carob germ extract is present in a concentration of between 0.0001% and 20% of the total weight of the composition, and preferably in a concentration of between 0.05% and 5% of the total weight of the composition, in a physiologically acceptable medium.

According to another advantageous embodiment of the invention, the active agent may be encapsulated or included in a cosmetic vector such as a liposome or any other microcapsule used in the field of cosmetics or adsorbed on powdery organic polymers, mineral supports such as talcs and bentonites.

The compositions for implementation of the invention may in particular be in the form of an aqueous, hydro-alcoholic or oily solution; and oil-in-water emulsion, a water-in-oil emulsion or multiple emulsions; they may also be in the form of suspensions, or powders, suitable for application on the skin, mucous membranes, lips and/or hair.

These compositions may be more or less fluid and have the appearance of a cream, a lotion, a milk, a serum, a pomade, a gel, a paste or a foam. They may also be in solid form such as a stick or be applied on the skin in the form of aerosol.

These compositions may also include any additive commonly used in the field of application envisaged, as well as the adjuvants necessary for their formulation, such as solvents, thickeners, diluents, antioxidants, coloring agents, sunscreens, self-tanning agents, pigments, fillers, preservatives, fragrances, odor absorbers, cosmetic or pharmaceutical active agents, essential oils, vitamins, essential fatty acids, surfactants, film-forming polymers, and so on.

In every case, a person skilled in the art will ensure that said adjuvants as well as the proportions thereof are chosen so as not to interfere with the desired advantageous properties of the composition of the invention. These adjuvants may, for example, correspond to 0.01 to 20% of the total weight of the composition. When the composition of the invention is an emulsion, the fatty phase may represent 5 to 80% by weight and preferably 5 to 50% by weight with respect to the total weight of the composition. The emulsifiers and co-emulsifiers used in the composition will be chosen from those conventionally used in the field considered. For example, they may be used in a proportion ranging from 0.3 to 30% by weight, with respect to the total weight of the composition.

Advantageously, the composition capable of being used for the invention may include, in addition to the slimming active agent according to the invention, at least one other active agent having cosmetic effects that are similar and/or complementary to those of the invention. According to the invention, this active agent will be defined as an “additional active agent”.

For example, the additional active agent(s) may be chosen from: anti-aging, toning, lightening, hydrating, draining, and microcirculation-promoting agents, pharmaceutical, exfoliating, scrubbing, extracellular matrix-stimulating, energy metabolism-activating, antibacterial, antifungal, calming, anti-free radical, anti-UV and anti-acne agents, anti-inflammatory agents, anesthetics, warming agents, cooling agents and weight-loss agents.

Such additional agents may be chosen from the groups including:
vitamin A and in particular retinoic acid, retinol, retinol propionate, retinol palmitate,
vitamin B3 and more specifically niacinamide, tocopherol nicotinate,
vitamin B5, vitamin B6, vitamin B12,
vitamin C, in particular ascorbic acid, ascorbyl glucoside, ascorbyl tetrapalmitate, magnesium and sodium ascorbyl phosphate,
vitamins E, F, H, K, PP, coenzyme Q10,
metalloproteinase inhibitors, a TIMP activator,
DHEA, precursors and derivatives thereof,
amino acids such as arginine, ornithine, hydroxypropline, hydroxyproline dipalmate, palmitoylglycine, hydroxylysine, methionine and derivatives thereof, N-acyl amino acid compounds,
natural or synthetic peptides, including di-, tri-, tetra-, penta- and hexapeptides and the lipophilic derivatives thereof, isomers and complexed with other species such as a metal ion (e.g. copper, zinc, manganese, magnesium, and others). For example, the peptides commercially known under the names MATRIXYL®, ARGIRELINE®, COLLAXYL™, PEPTIDE VINCI 02™, CHRONOGEN™, LAMINIXYL IS™, PEPTIDE Q10™,
plant-based peptide extracts such as extracts of soy, spelt, grapevine, rapeseed, linseed, rice, corn, pea,
yeast extracts, Artemia Salina extracts,
dehydroacetic acid (DHA),
phytosterols of synthetic or natural origin,
salicylic acid and derivatives thereof, alpha- and beta-hydroxyacids,
amino sugars, glucosamine, D-glucosamine, N-acetyl-glucosamine, N-acetyl-D-glycosamine, mannosamine, N-acetyl mannosamine, galactosamine, N-acetyl galactosamine,
extracts of polyphenols, isoflavones, flavonoids, such as grape extracts, pine extracts and olive extracts,
lipids such as ceramides or phospholipids, oils of animal origin, such as squalene or squalane; plant oils, such as sweet almond, copra, ricin, jojoba, olive, rapeseed, peanut, sunflower, wheat germ, corn germ, soy, cottonseed, alfalfa, poppy, winter squash, evening primrose, millet, barley, rye, safflower, passion fruit, hazelnut, palm, apricot seed, avocado, and calendula oil; ethoxylated plant oils, and shea butter,
all UV screens and sunscreens.

Especially advantageously, the invention may include at least one additional active agent known for its slimming effect action, inhibiting lipogenesis or stimulating lipolysis, such as: cyclic AMP and derivatives thereof, adenylate cyclase enzyme activating agents and phosphodiesterase enzyme inhibiting agents, centalla asiatica extract, asiaticoside and asiatic acid, methyl xanthines, thein, theophylline, theobromine, forskoline, esculin and esculoside, ACE inhibitors, the peptide Val-Trp, neuropeptide Y inhibitors, enkephalin, gingko biloba extract, dioscorea extract, rutin, verba mate extract, guarana extract, oligosaccharides, polysaccharides, carnitine, ivy extract, rockweed extract, hydrolyzed Prunella vulgaris extract, hydrolyzed Celosia cristata extract, Anogeissus leiocarpus extract, Manihot utilissima leaf extract, palmitoylcarnitine, carnosine, taurine, elderberry extract, algae extracts such as Palmaria Palmata extract, the synthetic peptide of sequence Arg-Gly-Ser-NH₂, sold under the name ATPEPTIDE™, the synthetic peptide of sequence Pro-Leu-Asp-Thr-Ala-Lys-Val-Arg-Leu-Gln sold under the name ATPEPTIDE™.

The composition capable of being used according to the invention may be applied by any suitable route, in particular oral or external topical, and the formulation of the compositions will be adapted by a person skilled in the art.

Advantageously, the compositions according to the invention are in a form suitable for topical application. These compositions must therefore contain a physiologically acceptable medium, i.e. compatible with the skin and skin appendages, and cover all cosmetic forms.

It is obvious that the invention concerns mammals in general, and more specifically human beings.

Specific embodiments of this cosmetic treatment method also result from the above description. Other advantages and features of the invention will be more apparent upon reading the examples provided for illustrative and non-limiting purposes.

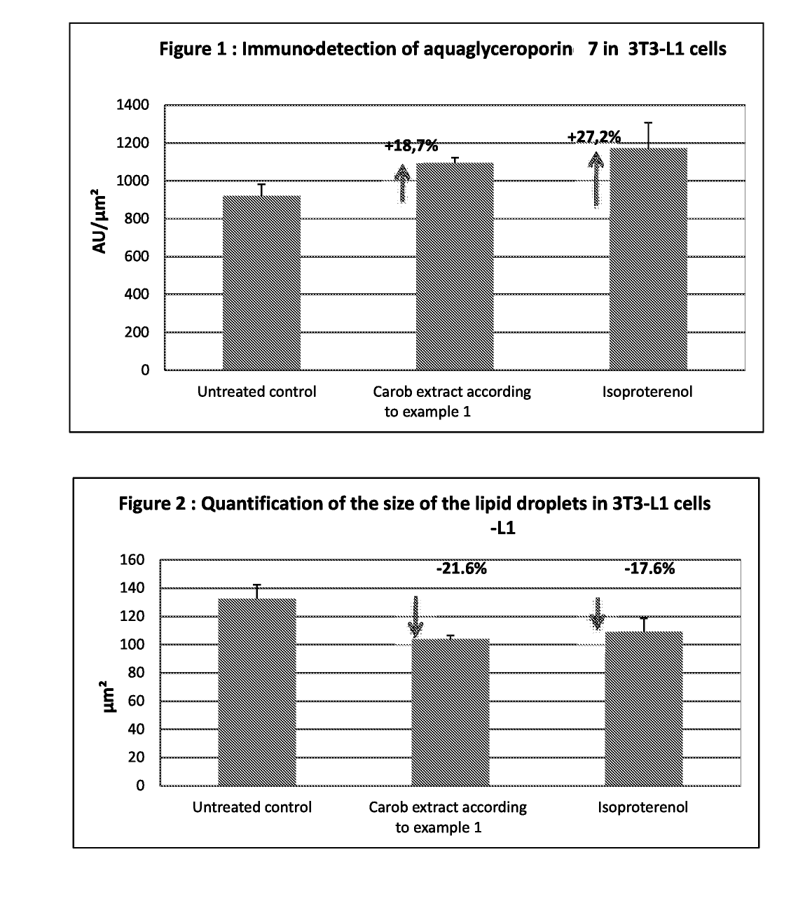

FIG. 1: Immuno-detection of aquaglyceroporin 7 in 3T3-L1 cells.

FIG. 2: Quantification of the size of lipid droplets in 3T3-L1 cells.

### EXAMPLE 1

### Preparation of a Carob Peptide Extract (Ceratonia siliqua L.)

Carob germ (Ceratonia siliqua L.) in powder form is placed in solution in 70 volumes of water and the pH is adjusted to a value of between 4.5 and 5.5.

To eliminate insoluble sugars, hydrolysis with a cellulase is performed. For this, 2% CELLUCLAST® CL (Novozymes) and 2% POLYCLAR® 10 (polyvinylpyrrolidone—PVPP—insoluble) are added to the reaction medium. The reaction medium is then heated for two hours at 50° C. then deactivated for one hour at 80° C. A filtration step makes it possible to separate the carbohydrate-rich filtrate so as to preserve only the solid residue.

The latter is characterized by a protein content of between 45 and 50% and a sugar content of between 20 and 30%.

The dry residue thus obtained is placed in solution in 100 volumes of water in the presence of 2% POLYCLAR® 10. The mixture is adjusted to a pH of between 8.0 and 8.5 with a 2 M aqueous soda solution.

To improve the extraction of proteins, a first hydrolysis is performed with 2% ALCALASE® (Novozymes). The hydrolysis is obtained after 2 hours, under stirring, at 55° C. The enzyme is inactivated by heating the solution at 80° C. for 2 hours. After deactivation, the reaction mixture is filtered and the filtrate is collected. This is the intermediate carob germ protein extract.

At this stage in the preparation, the peptide and protein compounds of said filtrate are characterized by electrophoresis on polyacrylamide gel (NUPAGE® Bis-Tris Precast gels, Invitrogen). For this, the filtrate is heated at 70° C. for 10 minutes under denaturing reducing conditions in a NUPAGE® LDS sample preparation buffer. A NUPAGE® antioxidant solution is added to the interior vat (cathode) in order to prevent the reduced proteins from being reoxidized during electrophoresis. The migration of proteins is performed in NUPAGE® MES migration buffer in the presence of a molecular weight standard (SeeBlue Plus2). The staining of the proteins is performed with COOMASSIE™ Blue R-250. The protein profile thus obtained shows that the peptide and protein compounds of the filtrate have molecular weights between 50 and 10 kDa.

The intermediate carob germ protein extract is then placed in solution in 100 volumes of water in the presence of 2% POLYCLAR® 10. The mixture is adjusted to a pH of between 4 and 5 with a 1 M aqueous hydrochloric acid solution.

A step of hydrolysis of the proteins is then performed with an endoprotease. For this, 2% bromelain is added to the reaction medium. The hydrolysis is obtained after 2 hours of stirring at 50° C. The enzyme is inactivated by heating the solution at 80° C. for 2 hours.

The purification of the extract thus obtained is continued by successive filtrations with Seitz-Orion filter-plates of decreasing porosity (to 0.2 μm) in order to obtain a clear, bright solution. After this filtration series, the carob germ extract is characterized by a dry weight between 20 and 25 g/kg, a protein content between 10 and 15 g/l, a sugar content between 5 and 6 g/l, an amino acid content between 1 and 2 g/l and a total polyphenol content between 0.5 and 1 g/l. The proteins are assayed by a specific colorimetric method (Lowry method).

The protein profile of this extract is analyzed by electrophoresis gel. Under the same conditions as described above, 2 large families of proteins are observed: the first family, in the minority, corresponds to proteins having a molecular weight of 25 to 20 kDa and the second family, very much in the majority, corresponds to proteins having a molecular weight below 5 kDa.

This extract is then purified by eliminating the proteins having a molecular weight above 5 kDa by tangential flow filtrations steps. For this, the carob germ extract is pumped under pressure through a Pellicon® substrate equipped with a Pellicon® 2 Biomax cassette 30 kDa. The first filtrate obtained is recovered so as to be filtered again through another Pellicon® 2 Biomax 5 kDa cassette.

At the end of the purification, a clear, bright, yellow-orange carob germ extract is obtained. It is characterized by a dry weight between 8 and 9 g/kg, a protein content between 6 and 7 g/l, a sugar content between 0.3 and 0.5 g/l and a total polyphenol content below 0.1 g/l.

Then, a dilution phase is performed in a water-glycerol mixture in order to obtain a peptide extract characterized by a dry weight of 2 to 5 g/kg, and preferably 2.5 g/kg, a peptide compound concentration of 1.5 to 3.5 g/l, a sugar concentration of 0.1 to 0.3 g/l (below 15%) and a polyphenol concentration below 1%.

This purified and diluted extract corresponds to the carob germ peptide extract according to the invention. It is characterized by the fact that the peptide compounds have a molecular weight below 5 kDa, with a polyphenol content below 1%.

This solution is then analyzed by high-pressure liquid chromatography with an HP1100 apparatus controlled by ChemStation software. The column used during elution of the carob extract is NUCLEOSIL® 300-5 C4 MPN (125×4 mn). This column makes it possible to chromatographically analyze proteins having molecular weights of 0.2 to 25 kDa (according to a suitable solvent gradient). Under these chromatographic conditions, a plurality of peptide fractions were isolated. These various fractions were analyzed by mass spectrometry in order to identify their molecular peaks. The amino acid composition was also determined. It is obtained after acid hydrolysis and identification by high-pressure liquid chromatography by means of pre-differentiation with PICT (phenyl-isothiocyanate).

### EXAMPLE 2

### Evaluation of the Expression of Aquaglyceroporin 7

The objective of this study is to determine the influence of the association of the carob extract according to example 1 and caffeine, on the expression of aquaglyceroporin 7 in differentiated 3T3-L1 adipocyte cells.

### Protocol:

Culture and differentiation of 3T3-L1 cells:

3T3-L1 adipocyte cells are cultivated in a DMEM medium with 4.5 g/l glucose, 2 mM glutamine and 10% fetal bovine serum.

Two days after the cell confluence phase, the differentiation of 3T3-L1 cells into adipocytes is induced by adding a solution of 0.5 mM of IBMX, 1 μM of dexamethasone and 10 μg/ml of insulin (Sigma, St. Louis, Mo., USA) in the culture medium for 3 days. Then, only the insulin is kept for 3 to 4 days of additional culture. The cells are then kept in culture, in a standard medium, for another 3 days.

Treatment:

The treatment is performed from the start of the differentiation induction phase by a daily application, for 12 days, of 1×PBS (Lonza, Rockland, Me., USA) for the untreated control, carob germ extract with a dry weight of 2.5 g/kg as obtained in example 1, diluted to 1% in PBS. A positive control is produced with a 5-hour treatment with 10 μM isoproterenol, which is an adrenergic receptor agonist, causing very rapid lipolysis.

Immunolabeling of Aquaglyceroporin 7:

The cells are washed 3 times with 1×PBS (Lonza, Rockland, Me., USA) and fixed with 3.7% formaldehyde (Sigma Aldrich, USA) for 10 minutes at ambient temperature. The cell membranes are permeabilized with acetone for 4 minutes at −20° C. The non-specific sites are saturated with 3% bovine serum albumin (Sigma-Aldrich, Steihheim, Germany) for 15 minutes. The primary antibody (rabbit polyclonal anti-aquaglyceroporin 7 (Santa Cruz Biotechnology) diluted to 1/100) is applied for 1.5 hour. After multiple rinses, a second antibody, coupled to the Alexa Fluor 488 probe (Alexa Fluor 488 donkey anti-rabbit (Invitrogen, Fisher) diluted to 1/1000) is applied for 1 hour. The slides are then mounted in the Fluoromount G (Electron Microscopy Science, Hatfield, UK).

The cells are examined with a Nikon Eclipse 80i microscope, with a 40× objective, and photographs are taken with the Nikon Digital DXM1200C camera.

Three images for each condition are analyzed using the Image-Pro Analyzer 6.3 software. The sum of light intensities is adjusted with respect to the culture surface (McMullen et al., 2010).

The statistical analysis uses a t-Student test for unpaired data, in which P<0.05 is considered to be significant; P<0.1 is considered to be very significant and P<0.005 is considered to be highly significant.

### Results:

The microscopic observations show a stronger fluorescence in the differentiated 3T3-L1 cells treated with the 1% carob germ extract. The quantitative analysis show an 18.7% increase with respect to the untreated control, as shown in FIG. 1.

### Conclusion:

The 1% carob extract according to example 1 causes a significant increase in the expression of aquaglyceroporin 7 in differentiated 3T3-L1 adipocyte cells.

### EXAMPLE 3

### Evaluation of Lipid Droplets Contained in Adipocytes

The objective of this study is to measure the influence of the association of the carob extract according to example 1 and caffeine, on the size and number of lipid droplets contained in the differentiated 3T3-L1 adipocyte cells.

### Protocol:

The culture, the differentiation of 3T3-L1 cells, then the treatment with the compounds to be tested are performed as in example 2.

Detection of Lipids by Nile Red:

The lipid detection is performed using Nile Red fluorescent stain, a phenoxazone that intensely labels the intracellular lipids.

The color of the fluorescence observed is directly dependent upon the hydrophobicity of the surrounding medium. This specific property of Nile Red makes it possible to differentiate neutral lipids, labeled in gold-yellow, from phospholipids, labeled in red.

The cells are fixed with a 3.7% formaldehyde solution for 10 minutes, then labeled by a solution of 100 nM Nile Red in PBS for 10 minutes, and finally rinsed in PBS.

The cells are examined with the fluorescence microscope. The quantification and statistical analysis are performed as in example 2.

### Results:

The intensity of the Nile Red labeling, the size and the number of droplets in the differentiated 3T3-L1 cells treated with the 1% carob extract according to example 1 is reduced with respect to the untreated cells.

The quantitative analyses show a 35.6% reduction in the intensity of the labeling, a 21.7% reduction in the size of the droplets and a 29% reduction in the number of droplets with respect to the untreated cells. This is shown in FIG. 2.

### Conclusion:

The 1% carob extract according to example 1 causes a significant reduction in the size and number of lipid droplets in differentiated 3T3-L1 adipocyte cells.

### EXAMPLE 4

### Preparation of Compositions

### 1 Weight-Loss Cream

| | | |
|---|---|---|
| Trade names | INCI names | Weight % |

Phase A

| | | |
|---|---|---|
| MONTANOV ® 68 | Cetearyl Alcohol (and) | 5.00 |
| | Cetearyl Glucoside | |
| SQUALENE ™ | Squalane | 2.50 |
| DUB IPP | Isopropyl Palmitate | 3.50 |
| EUTANOL ® G | Octyldodecanol | 1.50 |
| ROKONSAL ™ MEP | Phenoxyethanol (and) | 1 |
| | Methylparaben (and) | |
| | Ethylparaben (and) | |
| | Propylparaben | |

Phase B

| | | |
|---|---|---|
| Demineralized water | Aqua (Water) | qsf 100 |
| Glycerin | Glycerin | 3.00 |
| Butylene Glycol | Butylene glycol | 3.00 |

Phase C

| | | |
|---|---|---|
| SIMULGEL ® EG | Sodium Acrylate/ | 0.60 |
| | Acryloyldimethyl Taurate | |
| | Copolymer (and) | |
| | Isohexadecane (and) | |
| | Polysorbate 80 | |

Phase D

| | | |
|---|---|---|
| Carob extract according | | 1% |
| to example 1 | | |
| Fragrance | Fragrance | qsf |
| Coloring agent | | qsf |

### Procedure

The constituents of phase A are melted at 75° C. and the constituents of phase B are heated to 75° C. Phase A is emulsified with B, then the mixture is cooled below 40° C. Phases C and D are then added under constant stirring.

### 2 Firming—Slimming Spray

| | | |
|---|---|---|
| Trade names | INCI Names | Weight % |

Phase A

| | | |
|---|---|---|
| EMULGADE ® SEV | Glyceryl Stearate (and) | 4.60 |
| | Ceteareth-20 (and) | |
| | Ceteareth-12 (and) | |
| | Cetearyl Alcohol | |
| EUMULGIN ® B2 | Ceteareth-20 | 1.40 |
| CETIOL ® OE | Dicarpylyl Ether | 3.00 |
| DUB B1215 | C12-C15 Alkyl Benzoate | 5.00 |
| ROKONSOAL ™ MEP | Phenoxyethanol (and) | 1 |
| | Methylparaben (and) | |
| | Ethylparaben (and) | |
| | Propylparaben | |
| DUB ININ | Isononyl Isononanoate | 5.00 |

Phase B

| | | |
|---|---|---|
| Demineralized water | Aqua (Water) | 15.00 |
| Glycerin | Glycerin | 3.00 |

Phase C

| | | |
|---|---|---|
| Demineralized water | Aqua (Water) | qsf 100 |

Phase D

| | | |
|---|---|---|
| Carob extract according | | 3% |
| to example 1 | | |
| Fragrance | Fragrance | qsf |
| Coloring agent | | qsf |

### Procedure

The constituents of phase A and phase B are heated separately at 65° C.; phase B is incorporated with phase A under stirring. The temperature of the mixture is brought to 83° C., then cooled to the phase inversion temperature. Phase C is then added. Phase D is incorporated when the temperature falls below 40° C.

### 3 Firming—Slimming—Anti-Cellulite Gel

| Trade names | INCI Names | Weight % |
|---|---|---|
| CARBOPOL ® Ultrez 10 | Carbomer | 0.50 |
| Demineralized water | Aqua (Water) | qsf 100 |
| DUB DIOL | Methyl propanediol | 3.00 |
| EDTA | Tetrasodium EDTA | 0.10 |
| Liquid GERMALL ® plus | propylene glycol & | 0.50 |
| | diazolidinyl urea & | |
| | iodopropynyl | |
| | butylcarbamate | |
| Carob extract according to | | 2% |
| example 1 | | |
| Tea | Triethanolamine | 0.50 |
| Fragrance | Fragrance | qsf |
| Water-soluble coloring | | qsf |
| agent | | |

### Procedure

CARBOPOL® is dispersed in water until complete hydration. The ingredients are then added in the order indicated above, under stirring. The mixture is then neutralized with TEA. Fragrance and coloring agents are added if necessary.

1. A cosmetic method for obtaining a slimming effect comprising:
providing a composition containing a carob germ extract (Ceratonia siliqua L.) as a slimming active agent, and a physiologically acceptable medium; and
topically applying the composition on at least a portion of the skin of the body or face.

2. The method according to claim 1, wherein the carob germ extract is diluted in one or more physiologically acceptable solvents selected from the group consisting of water, glycerol, ethanol, propanediol, butylene glycol, dipropylene glycol, ethoxylated or propoxylated diglycols, cyclic polyols, and combinations thereof.

3. The method according to claim 1, wherein the carob germ extract contains between 1.5 and 3.5 g/l of peptide compounds.

4. The method according to claim 1, wherein the carob germ extract is a peptide extract wherein the peptide compounds have a molecular weight below 5 kDa.

5. The method according to claim 1, wherein topically applying comprises the application of an effective amount of the composition that increases the expression of aquaglyceroporins.

6. The method according to claim 1, wherein topically applying comprises the application of an effective amount of the composition that promotes lipolysis, the elimination of lipids and the export of glycerol from the adipocytes.

7. The method according to claim 1, wherein topically applying comprises the application of an effective amount of the composition that reduces localized excess body fat.

8. The method according to claim 1, wherein the carob germ extract is present in a concentration of between 0.0001% and 20% of the total weight of the composition.

9. The method according to claim 1, wherein the composition further comprises at least one additional active agent selected from the group consisting of vitamin A, retinoic acid, retinol, vitamin B3, vitamin B5, vitamin B6, vitamin B12, vitamin C, vitamin E, vitamin F, vitamin H, vitamin K, vitamin PP, coenzyme Q10, metalloproteinase inhibitors, amino acids, carnitine, carnosine, taurine, natural or synthetic peptides, plant-based peptides, yeast extracts, Artemia Salina extracts, phytosterols of synthetic or natural origin, salicylic acid, oligosaccharides, polysaccharides, amino sugars, polyphenols, flavonoids, lipids, phospholipids, cyclic AMP and derivatives thereof, adenylate cyclase enzyme activating agents, phosphodiesterase enzyme inhibiting agents, thein, theophylline, theobromine, forskoline, esculin, ACE inhibitors, dioscorea extract, guarana extract, ivy extract, rockweed extract, algae extract such as Palmata Palmaria, hydrolyzed Prunella vulgaris extract or elderberry extract.

10. The method according to claim 5, wherein the aquaglyceroporin is aquaglyceroporin 7.

11. The method according to claim 8, wherein the carob germ extract is present in a concentration of between 0.05% and 5% of the total weight of the composition.
